(19) United States
(12) Patent Application Publication
(10) Pub. No.: US 2006/0135932 A1
(43) Pub. Date: Jun. 22, 2006

(54) Stretchable absorbent core and wrap

(76) Inventors: Frank Paul Abuto, Duluth, GA (US); Hoa La Willhelm, Appleton, WI (US); Rob David Everett, Appleton, WI (US)

Correspondence Address: KIMBERLY-CLARK WORLDWIDE, INC., 401 NORTH LAKE STREET, NEENAH, WI, 54956, US

(21) Appl. No.: 11/020,842

(22) Filed: Dec. 21, 2004

(51) Int. Cl. A61F 13/15

(52) U.S. Cl. 604/385.22; 604/368

(57) ABSTRACT

A stretchable absorbent article comprises a stretchable backsheet and an absorbent core that is at least partially enveloped by a stretchable core wrap. The absorbent core has a quantity of superabsorbent materials contained within a matrix of polymer fibers. The stretchable core wrap has a mean flow pore diameter of less than about 41 microns. The stretchable article may additionally have a stretchable bodyside liner as well as other stretchable components. The stretchable absorbent article can provide greater performance as well as greater comfort and confidence among the user.

### BACKGROUND

Absorbent articles such as diapers, training pants, adult incontinence and feminine care products for receiving and retaining bodily discharges such as urine, menses and fecal matter are well known in the art, and a significant effort has been made to improve their performance, including fit and comfort. One such improvement concerns the development of thin and flexible absorbent articles.

For example, it may be desirable to utilize increasing amounts of superabsorbent materials and decreasing amounts of absorbent fibers in the absorbent core portion of such articles to help reduce the bulkiness of the articles. However, without the presence of a substantial matrix of fibers, the absorbent cores' integrity may be compromised. Therefore, it may be desirable to protect such absorbent cores with a core wrap.

At the same time, many absorbent articles now include stretchable backsheets or other stretchable components such as bodyside liners, leg elastics and waist elastics. However, such articles have also included non-stretchable absorbent components which can adversely affect the ability of the stretchable articles to function. This can also adversely affect the fit and comfort of the absorbent article, as well as the confidence of the user. Therefore, there is a desire for an absorbent article with improved performance, including improved fit and comfort.

### SUMMARY

The present invention concerns an absorbent article, suitably a disposable absorbent article, such as a training pant. Generally stated, the present invention provides a stretchable absorbent article which comprises a stretchable wrapped absorbent core having a high concentration of superabsorbent material. Specifically disclosed is an absorbent article which comprises at least a stretchable backsheet, an absorbent core comprising a quantity of superabsorbent materials, and a stretchable core wrap which has a mean flow pore diameter less than about 41 microns. This can result in greater performance of the article as well as greater comfort and confidence among the user.

Numerous other features and advantages of the present invention will appear from the following description. In the description, reference is made to the accompanying drawings which help illustrate exemplary embodiments of the invention. Such embodiments do not represent the full scope of the invention. Reference should therefore be made to the claims herein for interpreting the full scope of the invention.

### FIGURES

The foregoing and other features, aspects and advantages of the present invention will become better understood with regard to the following description, appended claims and accompanying drawings where:

FIG. 1 is a perspective view of one embodiment of an absorbent article that may be made in accordance with the present invention;

FIG. 2 is a plan view of the absorbent article shown in FIG. 1 with the article in an unfastened, unfolded and laid flat condition showing the surface of the article that faces the wearer when worn and with portions cut away to show underlying features;

FIG. 3 is a perspective view of an absorbent composite according to the present invention;

FIG. 4 is a cross-sectional side view of an absorbent composite according to the present invention;

FIG. 5 is a cross-sectional side view of another absorbent composite according to the present invention;

FIG. 6 is a cross-sectional side view of another absorbent composite according to the present invention;

FIG. 7 is a schematic diagram of one version of a method and apparatus for producing an absorbent core; and

FIG. 8 is a schematic side view of one version of a method and apparatus for forming an absorbent composite according to the present invention.

Repeated use of reference characters in the present specification and drawings is intended to represent the same or analogous features or elements of the present invention.

### DEFINITIONS

It should be noted that, when employed in the present disclosure, the terms “comprises,” “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

The term “absorbent article” generally refers to devices which can absorb and contain fluids. For example, personal care absorbent articles refer to devices which are placed against or near the skin to absorb and contain the various fluids discharged from the body. The term “disposable” is used herein to describe absorbent articles that are not intended to be laundered or otherwise restored or reused as an absorbent article after a single use. Examples of such disposable absorbent articles include, but are not limited to, personal care absorbent articles, health/medical absorbent articles, and household/industrial absorbent articles.

The term “coform” is intended to describe a blend of meltblown fibers and cellulose fibers that is formed by air forming a meltblown polymer material while simultaneously blowing air-suspended cellulose fibers into the stream of meltblown fibers. The coform material may also include other materials, such as superabsorbent materials. The meltblown fibers containing wood fibers are collected on a forming surface, such as provided by a foraminous belt. The forming surface may include a gas-pervious material, such as spunbonded fabric material, that has been placed onto the forming surface.

The terms “elastic,” “elastomeric” and “elastically extensible” are used interchangeably to refer to a material or composite that generally exhibits properties which approximate the properties of natural rubber. The elastomeric material is generally capable of being extended or otherwise deformed, and then recovering a significant portion of its shape after the extension or deforming force is removed.

The term “envelopes” refers to covering at least the entire bodyside surface of an absorbent core. The term “partially envelopes” refers to covering less than the entire bodyside surface of an absorbent core. The term “completely envelopes” refers to surrounding the entire absorbent core.

The term “extensible” refers to a material that is generally capable of being extended or otherwise deformed, but which does not recover a significant portion of its shape after the extension or deforming force is removed.

The term “fluid impermeable,” when used to describe a layer or laminate, means that fluid such as water or bodily fluids will not pass substantially through the layer or laminate under ordinary use conditions in a direction generally perpendicular to the plane of the layer or laminate at the point of fluid contact.

The term “health/medical absorbent article” includes a variety of professional and consumer health-care products including, but not limited to, products for applying hot or cold therapy, medical gowns (i.e., protective and/or surgical gowns), surgical drapes, caps, gloves, face masks, bandages, wound dressings, wipes, covers, containers, filters, disposable garments and bed pads, medical absorbent garments, underpads, and the like.

The term “household/industrial absorbent articles” include construction and packaging supplies, products for cleaning and disinfecting, wipes, covers, filters, towels, disposable cutting sheets, bath tissue, facial tissue, nonwoven roll goods, home-comfort products including pillows, pads, mats, cushions, masks and body care products such as products used to cleanse or treat the skin, laboratory coats, cover-alls, trash bags, stain removers, topical compositions, laundry soil/ink absorbers, detergent agglomerators, lipophilic fluid separators, and the like.

The terms “hydrophilic” and “wettable” are used interchangeably to refer to a material having a contact angle of water in air of less than 90 degrees. The term “hydrophobic” refers to a material having a contact angle of water in air of at least 90 degrees. For the purposes of this application, contact angle measurements are determined as set forth in Robert J. Good and Robert J. Stromberg, Ed., in “Surface and Colloid Science-Experimental Methods,” Vol. II, (Plenum Press, 1979), herein incorporated by reference in a manner consistent with the present disclosure.

The term “layer” when used in the singular can have the dual meaning of a single element or a plurality of elements.

The term “materials” when used in the phrase “superabsorbent materials” refers generally to discrete units. The units can comprise particles, granules, fibers, flakes, agglomerates, rods, spheres, needles, particles coated with fibers or other additives, pulverized materials, powders, films, and the like, as well as combinations thereof. The materials can have any desired shape such as, for example, cubic, rod-like, polyhedral, spherical or semi-spherical, rounded or semi-rounded, angular, irregular, etc. Additionally, superabsorbent materials may be composed of more than one type of material.

The term “meltblown fibers” refers to fibers formed by extruding a molten thermoplastic material through a plurality of fine, usually circular, die capillaries as molten threads or filaments into a high velocity, usually heated, gas (e.g., air) stream which attenuates the filaments of molten thermoplastic material to reduce their diameter. Thereafter, the meltblown fibers are carried by the high velocity gas stream and are deposited on a collecting surface to form a web of randomly disbursed meltblown fibers.

The terms “nonwoven” and “nonwoven web” refer to materials and webs of material having a structure of individual fibers or filaments which are interlaid, but not in an identifiable manner as in a knitted fabric. The terms “fiber” and “filament” are used herein interchangeably. Nonwoven fabrics or webs have been formed from many processes such as, for example, meltblowing processes, spunbonding processes, air laying processes, and bonded carded web processes. The basis weight of nonwoven fabrics is usually expressed in ounces of material per square yard (osy) or grams per square meter (gsm) and the fiber diameters are usually expressed in microns. (Note that to convert from osy to gsm, multiply osy by 33.91.)

The term “personal care absorbent article” includes, but is not limited to, absorbent articles such as diapers, diaper pants, baby wipes, training pants, absorbent underpants, child care pants, swimwear, and other disposable garments; feminine care products including sanitary napkins, wipes, menstrual pads, menstrual pants, panty liners, panty shields, interlabials, tampons, and tampon applicators; adult-care products including wipes, pads such as breast pads, containers, incontinence products, and urinary shields; clothing components; bibs; athletic and recreation products; and the like.

The term “polymers” includes, but is not limited to, homopolymers, copolymers, such as for example, block, graft, random and alternating copolymers, terpolymers, etc. and blends and modifications thereof. Furthermore, unless otherwise specifically limited, the term “polymer” shall include all possible configurational isomers of the material. These configurations include, but are not limited to isotactic, syndiotactic and atactic symmetries.

The terms “spunbond” and “spunbonded fiber” refer to fibers which are formed by extruding filaments of molten thermoplastic material from a plurality of fine, usually circular, capillaries of a spinneret, and then rapidly reducing the diameter of the extruded filaments.

The term “stretchable” refers to materials which may be extensible or which may be elastically extensible.

The terms “superabsorbent” and “superabsorbent materials” refer to a water-swellable, water-insoluble organic or inorganic materials capable, under the most favorable conditions, of absorbing at least about 10 times their weight, or at least about 15 times their weight, or at least about 25 times their weight in an aqueous solution containing 0.9 weight percent sodium chloride. Superabsorbent materials can be natural, synthetic, and modified natural polymers and materials. In addition, superabsorbent materials can be inorganic materials, such as silica gels, or organic compounds such as cross-linked polymers. Superabsorbent materials may be biodegradable, non-biodegradable, bipolar or ion-exchanged. Superabsorbent materials can also be incorporated in a structure by in-situ polymerization. In contrast, “absorbent materials” are capable, under the most favorable conditions, of absorbing at least 5 times their weight of an aqueous solution containing 0.9 weight percent sodium chloride.

The term “thermoplastic” refers to fibers which are formed from polymers such that the fibers can be bonded to themselves using heat or heat and pressure.

These terms may be defined with additional language in the remaining portions of the specification.

### DETAILED DESCRIPTION

The present invention concerns an absorbent article, suitably a disposable personal care absorbent article, such as a training pant. More particularly, the absorbent article comprises a stretchable backsheet, optionally a stretchable bodyside liner, an absorbent core, and a stretchable non-woven core wrap while at least partially envelopes the core, where the core wrap has a mean flow pore diameter less than about 41 microns. The result is an absorbent article which exhibits improved performance as well as greater comfort and confidence among the user.

In general, disposable absorbent articles typically include a backsheet, a fluid pervious bodyside liner joined to the backsheet, and an absorbent core positioned and held between the backsheet and the bodyside liner. An absorbent article may also include other components, such as fluid wicking layers, intake layers, surge layers, distribution layers, transfer layers, barrier layers, wrapping layers and the like, as well as combinations thereof.

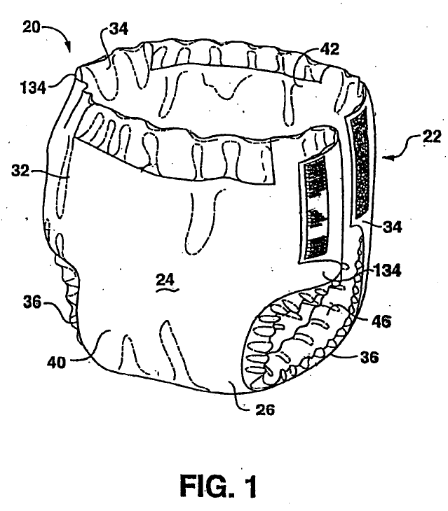

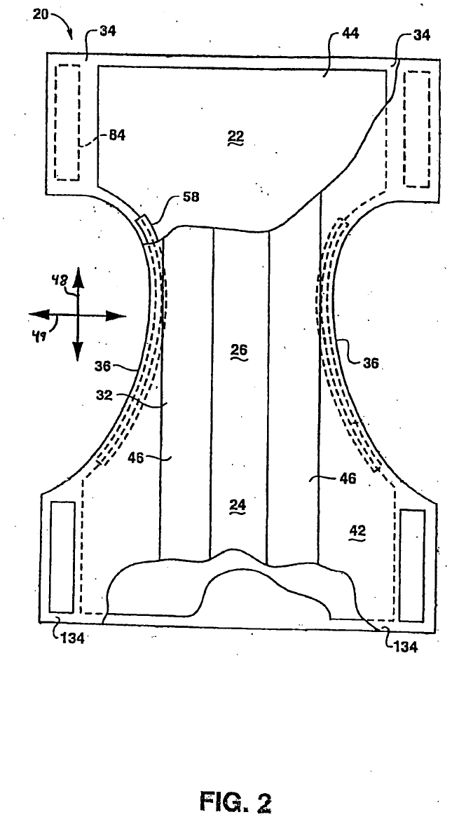

Referring to FIGS. 1 and 2 for exemplary purposes, a training pant which may incorporate the present invention is shown. It is understood that the present invention is suitable for use with various other absorbent articles, including but not limited to other personal care absorbent articles, pet care absorbent articles, health/medical absorbent articles, household/industrial absorbent articles, and the like without departing from the scope of the present invention.

Various materials and methods for constructing training pants are disclosed in PCT Patent Application WO 00/37009 published Jun. 29, 2000 by A. Fletcher et al; U.S. Pat. No. 4,940,464 to Van Gompel et al.; U.S. Pat. No. 5,766,389 to Brandon et al., and U.S. Pat. No. 6,645,190 to Olson et al., all of which are incorporated herein by reference in a manner that is consistent with the present disclosure.

FIG. 1 illustrates a training pant in a partially fastened condition, and FIG. 2 illustrates a training pant in an opened and unfolded state. The training pant defines a longitudinal direction that extends from the front of the training pant when worn to the back of the training pant. Perpendicular to the longitudinal direction is a lateral direction.

The pair of training pants defines a front region, a back region, and a crotch region extending longitudinally between and interconnecting the front and back regions. The pant also defines an inner surface adapted in use (e.g., positioned relative to the other components of the pant) to be disposed toward the wearer, and an outer surface opposite the inner surface. The training pant has a pair of laterally opposite side edges and a pair of longitudinally opposite waist edges.

The illustrated pant 20 may include a chassis 32, a pair of laterally opposite front side panels 34 extending laterally outward at the front region 22 and a pair of laterally opposite back side panels 134 extending laterally outward at the back region 24.

Referring to FIGS. 1 and 2, the chassis 32 includes a backsheet 40 and a bodyside liner 42 that may be joined to the backsheet 40 in a superimposed relation therewith by adhesives, ultrasonic bonds, thermal bonds or other conventional techniques. The chassis 32 may further include the absorbent composite 44 of the present invention such as shown in FIG. 2 disposed between the backsheet 40 and the bodyside liner 42 for absorbing fluid body exudates exuded by the wearer, and may further include a pair of containment flaps 46 secured to the bodyside liner 42 or the absorbent composite 44 for inhibiting the lateral flow of body exudates.

The backsheet 40, the bodyside liner 42 and the absorbent composite 44 may be made from many different materials known to those skilled in the art. All three layers, for instance, may be extensible and/or elastically extensible. Further, the stretch properties of each layer may vary in order to control the overall stretch properties of the product.

The backsheet 40, for instance, may be breathable and/or may be fluid impermeable. The backsheet 40 may be constructed of a single layer, multiple layers, laminates, spunbond fabrics, films, meltblown fabrics, elastic netting, microporous webs, or bonded carded webs. The backsheet 40, for instance, can be a single layer of a fluid impermeable material, or alternatively can be a multi-layered laminate structure in which at least one of the layers is fluid impermeable.

The backsheet 40 can be biaxially extensible and optionally biaxially elastic. Elastic non-woven laminate webs that can be used as the backsheet 40 include a non-woven material joined to one or more gatherable non-woven webs, or films. Stretch Bonded Laminates (SBL) and Neck Bonded Laminates (NBL) are examples of elastomeric composites.

Examples of suitable nonwoven materials are spunbond-meltblown fabrics, spunbond-meltblown-spunbond fabrics, spunbond fabrics, or laminates of such fabrics with films, or other nonwoven webs. Elastomeric materials may include cast or blown films, meltblown fabrics or spunbond fabrics composed of polyethylene, polypropylene, or polyolefin elastomers, as well as combinations thereof. The elastomeric materials may include PEBAX elastomer (available from AtoFina Chemicals, Inc., a business having offices located in Philadelphia, Pa. U.S.A), HYTREL elastomeric polyester (available from Invista, a business having offices located in Wichita, Kans. U.S.A.), KRATON elastomer (available from Kraton Polymers, a business having offices located in Houston, Tex., U.S.A.), or strands of LYCRA elastomer (also available from Invista), or the like, as well as combinations thereof. The backsheet 40 may include materials that have elastomeric properties through a mechanical process, printing process, heating process, or chemical treatment. For example, such materials may be apertured, creped, neck-stretched, heat activated, embossed, and micro-strained; and may be in the form of films, webs, and laminates.

One example of a suitable material for a biaxially stretchable backsheet 40 is a breathable elastic film/nonwoven laminate, such as described in U.S. Pat. No. 5,883,028, to Morman et al., incorporated herein by reference in a manner that is consistent with the present disclosure. Examples of materials having two-way stretchability and retractability are disclosed in U.S. Pat, No. 5,116,662 to Morman and U.S. Pat, No. 5,114,781 to Morman, all of which are incorporated herein by reference in a manner that is consistent with the present disclosure. These two patents describe composite elastic materials capable of stretching in at least two directions. The materials have at least one elastic sheet and at least one necked material, or reversibly necked material, joined to the elastic sheet at least at three locations arranged in a nonlinear configuration, so that the necked, or reversibly necked, web is gathered between at least two of those locations.

The bodyside liner 42 is suitably compliant, soft-feeling, and non-irritating to the wearer's skin. The bodyside liner 42 is also sufficiently liquid permeable to permit liquid body exudates to readily penetrate through its thickness to the absorbent composite 44. A suitable bodyside liner 42 may be manufactured from a wide selection of web materials, such as porous foams, reticulated foams, apertured plastic films, woven and non-woven webs, or a combination of any such materials. For example, the bodyside liner 42 may include a meltblown web, a spunbonded web, or a bonded-carded-web composed of natural fibers, synthetic fibers or combinations thereof. The bodyside liner 42 may be composed of a substantially hydrophobic material, and the hydrophobic material may optionally be treated with a surfactant or otherwise processed to impart a desired level of wettability and hydrophilicity.

The bodyside liner 42 may also be extensible and/or elastomerically extensible. Suitable elastomeric materials for construction of the bodyside liner 42 can include elastic strands, LYCRA elastics, cast or blown elastic films, nonwoven elastic webs, meltblown or spunbond elastomeric fibrous webs, as well as combinations thereof. Examples of suitable elastomeric materials include KRATON elastomers, HYTREL elastomers, ESTANE elastomeric polyurethanes (available from Noveon, a business having offices located in Cleveland, Ohio U.S.A.), or PEBAX elastomers. The bodyside liner 42 can also be made from extensible materials such as those described in U.S. Pat. No. 6,552,245 to Roessler et al. which is incorporated herein by reference in a manner that is consistent with the present disclosure. The bodyside liner 42 can also be made from biaxially stretchable materials as described in U.S. Pat. No. 6,641,134 filed to Vukos et al. which is incorporated herein by reference in a manner that is consistent with the present disclosure.

The article 20 can optionally further include a surge management layer which may be located adjacent the absorbent composite 44 and attached to various components in the article 20 such as the absorbent composite 44 or the bodyside liner 42 by methods known in the art, such as by using an adhesive. In general, a surge management layer helps to quickly acquire and diffuse surges or gushes of liquid that may be rapidly introduced into the absorbent structure of the article. The surge management layer can temporarily store the liquid prior to releasing it into the storage or retention portions of the absorbent composite 44. Examples of suitable surge management layers are described in U.S. Pat. No. 5,486,166 to Bishop et al.; U.S. Pat. No. 5,490,846 to Ellis et al.; and U.S. Pat. No. 5,820,973 to Dodge et al, all of which are incorporated herein by reference in a manner that is consistent with the present disclosure.

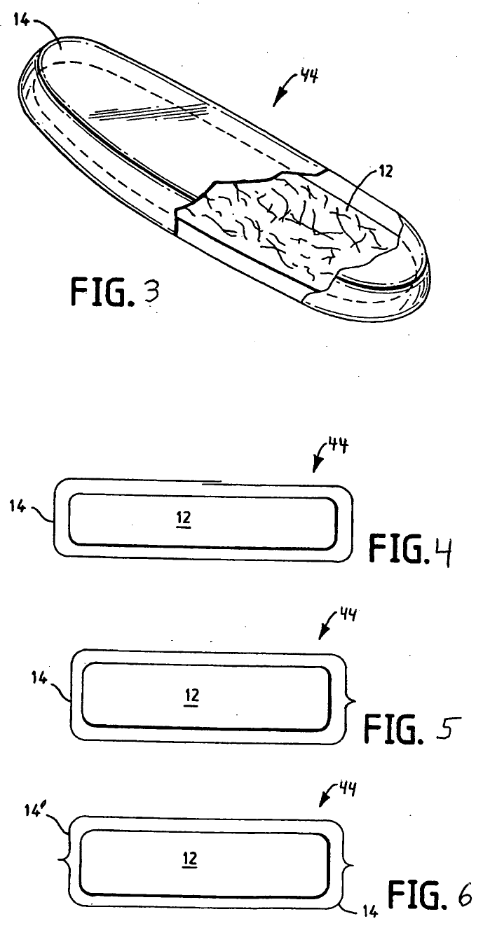

The article 20 can further comprise the absorbent composite 44 of the present invention. With additional reference to FIGS. 3-6, the absorbent composite 44 can include a stretchable absorbent core 12 component at least partially enveloped in a stretchable core wrap 14. The absorbent composite 44 can be attached to an absorbent article by bonding means known in the art, such as ultrasonic, pressure, adhesive, aperturing, heat, sewing thread or strand, autogenous or self-adhering, hook-and-loop, or any combination thereof. In addition, in some aspects of the invention, a portion of the stretchable core wrap 14 can also function as a bodyside liner 42, thus eliminating the need for a separate liner. Likewise, a portion of the stretchable core wrap 14 can also function as a moisture barrier (not shown), thus eliminating the need for a separate moisture barrier.

In general, the absorbent core 12 can have a significant amount of stretchability. For example, the absorbent core 12 can comprise a matrix of fibers which includes an operative amount of elastomeric polymer fibers. Other methods known in the art can include attaching superabsorbent particles to a stretchable film, utilizing a nonwoven substrate having cuts or slits in its structure, and the like.

The absorbent core 12 can also include absorbent material, such as superabsorbent material and/or fluff. Additionally, the superabsorbent material can be operatively contained within a matrix of fibers. Accordingly, the absorbent composite 44 can comprise a stretchable absorbent core 12 that includes a quantity of superabsorbent material and/or fluff contained within a matrix of fibers. In some aspects, the amount of superabsorbent material in the absorbent core 12 can be at least about 40-percent by weight of the core, such as at least about 60-percent or at least about 80-percent by weight of the core to provide improved benefits. Optionally, the amount of superabsorbent material can be at least about 95-percent by weight of the core. In other aspects, the absorbent core 12 can comprise about 35-percent or less by weight fluff fiber, such as about 25-percent or less, or 15-percent or less by weight fluff fiber.

It should be understood that the present invention is not restricted to use with superabsorbent materials and/or fluff. In some aspects, the absorbent core 12 may additionally or alternatively include materials such as surfactants, ion exchange resin particles, moisturizers, emollients, perfumes, natural fibers, synthetic fibers, fluid modifiers, odor control additives, and combinations thereof. Alternatively, the absorbent core 12 can be or can include a foam.

The absorbent core 12 may have any of a number of shapes. For example, it may have a 2-dimensional or 3-dimensional configuration, and may be rectangular shaped, triangular shaped, oval shaped, race-track shaped, I-shaped, generally hourglass shaped, T-shaped and the like. It is often suitable for the absorbent core 12 to be narrower in the crotch portion 36 than in the rear 34 or front 32 portion(s).

In order to function well, the absorbent composite 44 of the-present invention can have certain desired properties to provide improved performance as well as greater comfort and confidence among the user. For instance, the components of the absorbent composite 44 can have corresponding configurations of absorbent capacities, densities, basis weights and/or sizes which are selectively constructed and arranged to provide desired combinations of absorbency properties such as liquid intake rate, absorbent capacity, liquid distribution, or fit properties such as shape maintenance and aesthetics. Likewise, the components can have desired wet to, dry strength ratios, mean flow pore sizes, permeabilities, and elongation values.

For instance, the absorbent core 12 of the present invention can have selected densities as determined under a confining pressure of 0.05 psi (0.345 KPa). In some aspects, the absorbent core density can be at least a minimum of about 0.1 grams per cubic centimeter (g/cm³). The density of the absorbent core can alternatively be at least about 0.25 g/cm³, and can optionally be at least about 0.3 g/cm. In another feature, the density of the absorbent core can be up to about 0.4 g/cm³. Particular aspects or portions of the absorbent core can have a density within the range of about 0.20 to 0.35 g/cm³.

In another example, the absorbent core 12 can have desirable basis weights. In one feature, the absorbent core can have a basis weight of at least about 200 grams per square meter (gsm). In another feature, the basis weight of the absorbent core can be at least about 800 gsm. In still another feature, the basis weight of the absorbent core can be at least about 1200 gsm.

In yet another example, the absorbent core 12 can have desirable stretchable properties. In some aspects, the absorbent core 12, while in a dry state, can be extensible, and/or elastomerically extensible at least about 30-percent, such as at least about 50-percent, or at least about 75-percent, based on length in an unstretched condition. Alternatively, the absorbent components of the present invention can be extensible, and/or elastomerically extensible at about 200-percent or less, such as about 100-percent or less based on length in an unstretched condition to provide desired effectiveness.

If the stretchability parameter is outside the desired values, the absorbent core may not be sufficiently flexible to provide desired levels of fit and conformance to the shape of the user. A donning of a product that includes such an absorbent core would then be more difficult. For example, training pant products may be accidentally stretched to large amounts before use, and the absorbent system may rip and tear. As a result, the absorbent core may exhibit excessive leakage problems.

The stretchable core wrap 14 is particularly well-suited for enveloping and/or containing stretchable absorbent cores which are made at least partially from particulate matter such as superabsorbent materials. Accordingly, the core wrap 14 may envelope, partially envelope, or completely envelope the stretchable absorbent core 12. The core wrap 14 can include any porous polymeric films, nonwoven materials and combinations thereof known in the art. For example, in some aspects, the core wrap 14 can comprise meltblown, spunbond, spunlace, spunbond-meltblown-spunbond, coform, or combinations thereof As with the absorbent core 12, the core wrap 14 may also have a significant amount of stretchability. For example, the structure of the core wrap 14 can include an operative amount of elastomeric polymer fibers. Furthermore, the fibers utilized in the core wrap 14 can be continuous or discontinous.

In one aspect, the core wrap 14 can comprise a stretchable, durable, hydrophilic, fluid pervious substrate. In a further feature, the substrate can comprise a coating including a hydrophilicity boosting amount of nanoparticles, wherein such nanoparticles have a particle size of from 1 to 750 nanometers. Examples of suitable nanoparticles include titanium dioxide, layered clay minerals, alumina oxide, silicates, and combinations thereof. Optionally, a nonionic surfactant can be added to such core wrap to provide additional or enhanced benefits.

In another aspect of the present invention, the core wrap 14 can be treated with a high-energy surface treatment. This high-energy treatment may be prior to or concurrent with the hydrophilicity boosting composition coating described above. The high-energy treatment may be any suitable high-energy treatment for increasing the hydrophilicity of the core wrap. Suitable high-energy treatments, include but are not limited to, corona discharge treatment, plasma treatment, UV radiation, ion beam treatment, electron beam treatment and combinations thereof.

In still other aspects, the stretchable core wrap 14 may include absorbent materials, such as superabsorbent materials and/or absorbent fibers, such as fluff fibers, which make the core wrap absorbent. Such materials can be bonded directly to a surface of the core wrap 14 using methods known in the art, such as hot melt adhesive bonding, or such materials may be incorporated into the structure of the core wrap 14 during a manufacturing process, such as in a coform process. In yet other aspects, the core wrap 14 may additionally or alternatively include materials such as surfactants, ion exchange resin particles, moisturizers, emollients, perfumes, natural fibers, synthetic fibers, fluid modifiers, odor control additives, lotions, viscosity modifiers, anti-adherence agent, pH control agents, and the like, and combinations thereof.

It is also within the scope of the present invention that the core wrap 14 may be in the form of films, nonwoven webs, and laminates of two or more substrates or webs. Additionally, the core wrap 14 may be textured, apertured, creped, neck-stretched, heat activated, embossed, and micro-strained. Care must be taken when using apertured core wrap materials to wrap absorbent cores containing superabsorbent materials or other particulate materials. The apertures must not be too large as the materials may escape from the absorbent core. The size of such aperatures will be dependent upon the size of the materials utilized. In general, the aperature size should be smaller than the material size.

Similar to the absorbent core 12, the core wrap 14 of the present invention is also specifically designed and engineered to provide improved performance as well as greater comfort and confidence among the user. For instance, the stretchable core wrap 14 of the present invention can have selected wet to dry strength ratios. In some aspects, the core wrap 14 can have a wet to dry strength ratio above 0.5 and sometimes 1.0 or higher.

In another example, the core wrap can have desirable air permeabilities. In one aspects, the core wrap 14 can have an air permeability of 200 cubic meters per square meter per minute or greater as measured by the Air Permeability Test described below. In other aspects, the core wrap can have an air permeability in the range of 200 to 3500 cubic meters per square meter per minute. In one particular example, the core wrap has an air permeability of 235 cubic meters per square meter per minute. In another particular example, the core wrap has an air permeability of 3495 cubic meters per square meter per minute.

In another instance, the stretchable core wrap 14 can have desirable mean flow pore diameters. In general, the stretchable core wrap of the present invention should have a mean flow pore diameter that is less than about 41 microns as measured by the Mean Flow Pore Diameter Test described below. In some aspects, the core wrap can have a mean flow pore diameter in the range of about 5 to about 35 microns. In one particular example, the core wrap has a mean flow pore diameter of 34.7 microns. In another particular example, the core wrap has a mean flow pore diameter of 7.8 microns. It may be suitable in some aspects that less than about 5% of the total pores for any given area of the core wrap should have a mean flow pore diameter of about 50 microns or greater. More suitably, less than about 1% of the total pores for a given area should have a mean flow pore diameter of about 50 microns or greater.

In still another instance, the stretchable core wrap 14 can have desired basis weights. In some aspects, the core wrap can have a basis weight that is less than about 200 gsm. In other aspects, the core wrap can have a basis weight in the range of about 5 to about 120 gsm.

In yet another instance, the core wrap 14 of the present invention can have desirable stretchability properties. In general, once the absorbent core 12 has been wrapped with the core wrap 14, the core wrap 14 should have the ability to stretch in conjunction with the absorbent core, or with other various components of the stretchable article 20. In one particular aspect, the core wrap 14 is co-extensive with the absorbent core 12. While in a dry state, the core wrap 14 can be extensible, and/or elastomerically extensible at least about 30-percent, such as at least about 60-percent, or at least about 90-percent in the machine direction (MD), and at least about 50%, such as at least about 100%, or at least about 300% in the cross-machine direction (CD), based on length in an unstretched condition. Alternatively, the core wrap can have an MD elongation in the range of about 30% to about 200%, and a CD elongation of about 50% to about 700%. In one particular example, the core wrap has an MD elongation of 61.4% when a biasing force of 765.5 grams is applied, as measured by the Elongation Test described below. In another particular example, the core wrap has an MD elongation of 103.8% when a biasing force of 3081.9 grams is applied. In still another particular example, the core wrap has a CD elongation of 346.1% when a biasing force of 280.2 grams is applied. In yet another particular example, the core wrap has a CD elongation of 620.9% when a biasing force of 2218.9 grams is applied.

The core wrap 14 can also have a desirable elastic recovery which determines the amount or portion of the core wrap's shape that is recovered after an extension or deforming force is removed. In some aspects, the core wrap can recover at least about 1% of its shape in either the MD or the CD direction. In other aspects, the core wrap can recover less than about 99% of its shape in either the MD or the CD direction. In one particular aspect, the core wrap has an elastic recovery between about 89% and about 95% in the MD direction as measured by the Cycle Elastic Recovery Test described below. In another particular aspect, the core wrap has an elastic recovery between about 23% and about 66% in the CD direction.

In still another example, the core wrap 14 can have desirable fiber diameters. In some aspects, an operative amount of the polymer fibers in the core wrap 14 can have a fiber diameter of about 20 μm or less, such as about 8 μm or less, or about 7 μm or less. By way of example only, some aspects can comprise at least about 80% by weight, polymer fibers having a diameter of 8 μm or less. In other aspects, the core wrap can comprise at least about 95% by weight polymer fibers having a diameter of 7 μm or less.

As referenced above, at least one component of the absorbent composite 44 (i.e., the absorbent core and/or the core wrap) can optionally comprise a desired quantity of absorbent fibers, such as fluff fibers. Such fibers include cellulosic or other hydrophilic fibers which are utilized in the absorbent composite 44 to, among other things, help provide increased levels of fluid intake and wicking. Excessive amounts of such fibers, however, can undesirably increase the caliper of the composite and may limit properties such as extensibility, elasticity, and recovery. Additionally, overly large amounts of such fibers can lead to cracking of the absorbent composite 44 during stretching.

The cellulosic fibers may include, but are not limited to, chemical wood pulps such as sulfite and sulfate (sometimes called Kraft) pulps, as well as mechanical pulps such as ground wood, thermomechanical pulp and chemithermomechanical pulp. More particularly, the pulp fibers may include cotton, other typical wood pulps, cellulose acetate, debonded chemical wood pulp, and combinations thereof. Pulps derived from both deciduous and coniferous trees can be used. Additionally, the cellulosic fibers may include such hydrophilic materials as natural plant fibers, milkweed floss, cotton fibers, microcrystalline cellulose, microfibrillated cellulose, or any of these materials in combination with wood pulp fibers. Suitable cellulosic fibers can, for example, include NB 416, a bleached southern softwood Kraft pulp, available from Weyerhaeuser Co., a business having offices located in Federal Way, Wash. U.S.A.; CR 54, a bleached southern softwood Kraft pulp, available from Bowater Inc., a business having offices located in Greenville, S.C. U.S.A.; SULPHATATE HJ, a chemically modified hardwood pulp, available from Rayonier Inc., a business having offices located in Jesup, Ga. U.S.A.; NF 405, a chemically treated bleached southern softwood Kraft pulp, available from Weyerhaeuser Co.; and CR 1654, a mixed bleached southern softwood and hardwood Kraft pulp, available from Bowater Inc.

As referenced above, at least one of the components of the absorbent composite 44 may also include a desired amount of superabsorbent material. The superabsorbent material can be selected from natural, synthetic and modified natural polymers and materials. The superabsorbent material can be inorganic materials, such as silica gels, or organic compounds, such as crosslinked polymers. The term “crosslinked” refers to any means for effectively rendering normally water-soluble materials substantially water insoluble, but swellable. Such means can comprise, for example, physical entanglement, crystalline domains, covalent bonds, ionic complexes and associations, hydrophilic associations, such as hydrogen bonding, and hydrophobic associations or Van der Waals forces. The superabsorbent material can also be modified, such as by surface treating with a cross-linking, substantially non-covalently bonded surface coating with a partially hydrolysable cationic polymer, such as that disclosed in recently filed U.S. patent application Ser. No. 10/631,916 entitled “Absorbent Materials And Absorbent Articles Incorporating Such Absorbent Materials” filed Jul. 31, 2003 by Qin et al., which is incorporated herein by reference in a manner that is consistent with the present disclosure.

Examples of synthetic, polymeric, superabsorbent materials include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrolidone), poly(vinyl morpholinone), poly(vinyl alcohol), and mixtures and copolymers thereof. Further polymers suitable for use in the absorbent composite 44 include natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthum gum, locust bean gum, and the like. Mixtures of natural and wholly or partially synthetic absorbent polymers can also be useful. Processes for preparing synthetic, absorbent gelling polymers are disclosed in U.S. Pat. No. 4,076,663, to Masuda et al. and U.S. Pat. No. 4,286,082, to Tsubakimoto et al., all of which are incorporated herein by reference in a manner that is consistent with the present disclosure.

Superabsorbent materials suitable for use in the present invention are known to those skilled in the art. Generally stated, the superabsorbent material can be a water-swellable, generally water-insoluble, hydrogel-forming polymeric absorbent material, which is capable, under the most favorable conditions, of absorbing at least about 10 times its weight, or at least about 15 times its weight, or at least about 25 times its weight in an aqueous solution containing 0.9 weight percent sodium chloride. The hydrogel-forming polymeric absorbent material may be formed from organic hydrogel-forming polymeric material, which may include natural material such as agar, pectin, and guar gum; modified natural materials such as carboxymethyl cellulose, carboxyethyl cellulose, chitosan salt, and hydroxypropyl cellulose; and synthetic hydrogel-forming polymers. Synthetic hydrogel-forming polymers include, for example, alkali metal salts of polyacrylic acid, polyacrylamides, polyvinyl alcohol, ethylene maleic anhydride copolymers, polyvinyl ethers, polyvinyl morpholinone, polymers and copolymers of vinyl sulfonic acid, polyacrylates, polyvinyl amines, polyquaternary ammonium, polyacrylamides, polyvinyl pyridine, and the like. Other suitable hydrogel-forming polymers include hydrolyzed acrylonitrile grafted starch, acrylic acid grafted starch, and isobutylene maleic anhydride copolymers and mixtures thereof. The hydrogel-forming polymers are desirably lightly crosslinked to render the material substantially water insoluble. Crosslinking may, for example, be by irradiation or covalent, ionic, Van der Waals, or hydrogen bonding. Suitable base superabsorbent materials are available from various commercial vendors, such as Stockhausen, Inc., BASF Inc. and others. In one particular aspect, the superabsorbent material is FAVOR SXM 9394, available from Stockhausen, Inc., a business having offices located in Greensboro, N.C., U.S.A. The superabsorbent material may desirably be included in an appointed storage or retention portion of the absorbent system, and may optionally be employed in other components or portions of the absorbent article. In one feature, the superabsorbent material can be selectively positioned within the composite such that the absorbent core comprises regions of varying superabsorbent material concentration. Superabsorbent materials can be incorporated externally or by in-situ polymerization.

As mentioned above, the components of the absorbent composite 44 can include elastomeric polymer fibers. The elastomeric material of the polymer fibers may include an olefin elastomer or a non-olefin elastomer, as desired. For example, the elastomeric fibers can include olefinic copolymers, polyethylene elastomers, polypropylene elastomers, polyester elastomers, polyisoprene, cross-linked polybutadiene, diblock, triblock, tetrablock, or other multi-block thermoplastic elastomeric and/or flexible copolymers such as block copolymers including hydrogenated butadiene-isoprene-butadiene block copolymers; stereoblock polypropylenes; graft copolymers, including ethylene-propylene-diene terpolymer or ethylene-propylene-diene monomer (EPDM) rubber, ethylene-propylene random copolymers (EPM), ethylene propylene rubbers (EPR), ethylene vinyl acetate (EVA), and ethylene-methyl acrylate (EMA); and styrenic block copolymers including diblock and triblock copolymers such as styrene-isoprene-styrene (SIS), styrene-butadiene-styrene (SBS), styrene-isoprene-butadiene-styrene (SIBS), styrene-ethylene/butylene-styrene (SEBS), or styrene-ethylene/propylene-styrene (SEPS), which may be obtained from Kraton Inc., a business having offices located in Houston, Tex. U.S.A. under the trade designation KRATON elastomeric resin or from Dexco, a division of ExxonMobil Chemical Company, a business having offices located in Houston, Tex. U.S.A. under the trade designation VECTOR (SIS and SBS polymers); blends of thermoplastic elastomers with dynamic vulcanized elastomer-thermoplastic blends; thermoplastic polyether ester elastomers; ionomeric thermoplastic elastomers; thermoplastic elastic polyurethanes, including those available from Invista Corporation under the trade name LYCRA polyurethane, and ESTANE available from Noveon, Inc., a business having offices located in Cleveland, Ohio U.S.A; thermoplastic elastic polyamides, including polyether block amides available from AtoFina Chemicals, Inc., a business having offices located in Philadelphia, Pa. U.S.A. under the trade name PEBAX; polyether block amide; thermoplastic elastic polyesters, including those available from E. I. Du Pont de Nemours Co., under the trade name HYTREL, and ARNITEL from DSM Engineering Plastics, a business having offices located in Evansville, Ind., U.S.A. and single-site or metallocene-catalyzed polyolefins having a density of less than about 0.89 grams/cubic centimeter, available from Dow Chemical Co., a business having offices located in Freeport, Tex. U.S.A. under the trade name AFFINITY; and combinations thereof.

As used herein, a tri-block copolymer has an ABA structure where the A represents several repeat units of type A, and B represents several repeat units of type B. As mentioned above, several examples of styrenic block copolymers are SBS, SIS, SIBS, SEBS, and SEPS. In these copolymers the A blocks are polystyrene and the B blocks are a rubbery component. Generally these triblock copolymers have molecular weights that can vary from the low thousands to hundreds of thousands and the styrene content can range from 5-percent to 75-percent based on the weight of the triblock copolymer. A diblock copolymer is similar to the triblock but is of an AB structure. Suitable diblocks include styrene-isoprene diblocks, which have a molecular weight of approximately one-half of the triblock molecular weight having the same ratio of A blocks to B blocks.

In desired arrangements, the polymer fibers can include at least one material selected from the group consisting of styrenic block copolymers, elastic polyolefin polymers and co-polymers and EVA/AMA type polymers.

In other particular arrangements, for example, the elastomeric material of the polymer fibers can include various commercial grades of low crystallinity, lower molecular weight metallocene polyolefins, available from ExxonMobil Chemical Company, a company having offices located in Houston, Tex., U.S.A. under the VISTAMAXX trade designation. The VISTAMAXX material is believed to be metallocene propylene ethylene co-polymer. In one example, the elastomeric polymer was VISTAMAXX PLTD 2210. In other aspects, the elastomeric polymer can be VISTAMAXX PLTD 1778. Another optional elastomeric polymer is KRATON blend G 2755 from Kraton Inc. The KRATON material is believed to be a blend of styrene ethylene-butylene styrene polymer, ethylene waxes and tackifying resins.

In some aspects, the elastomeric polymer fibers can be produced from a polymer material having a selected melt flow rate (MFR). In a particular aspect, the MFR can be up to a maximum of about 300. Alternatively, the MFR can be up to about 230 or 250. In another aspect, the MFR can be a minimum of not less than about 20. The MFR can alternatively be not less than about 50 to provide desired performance. The described melt flow rate has the units of grams flow per 10 minutes (g/10 min). The parameter of melt flow rate is well known and can be determined by conventional techniques, such as by employing test ASTM D 1238 70 “extrusion plastometer” Standard Condition “L” 230° C. and 2.16 kg applied force.

As mentioned above, the polymer fibers of the absorbent core 12 and/or the core wrap 14 can include an amount of a surfactant. The surfactant can be combined with the polymer fibers in any operative manner. Various techniques for combining the surfactant are conventional and well known to persons skilled in the art. For example, the surfactant may be compounded with the polymer employed to form a meltblown fiber structure. In a particular feature, the surfactant may be configured to operatively migrate or segregate to the outer surface of the fibers upon the cooling of the fibers. Alternatively, the surfactant may be applied to or otherwise combined with the polymer fibers after the fibers have been formed.

The polymer fibers can include an operative amount of surfactant, based on the total weight of the fibers and surfactant. In some aspects, the polymer fibers can include at least a minimum of about 0.1 -percent by weight surfactant, as determined by water extraction. The amount of surfactant can alternatively be at least about 0.15-percent by weight, and can optionally be at least about 0.2-percent by weight to provide desired benefits. In other aspects, the amount of surfactant can be generally not more than a maximum of about 2-percent by weight, such as not more than about 1-percent by weight, or not more than about 0.5-percent by weight to provide improved performance.

If the amount of surfactant is outside the desired ranges, various disadvantages can occur. For example, an excessively low amount of surfactant may not allow fibers, such as hydrophobic meltblown fibers, to wet with the absorbed fluid. In contrast, an excessively high amount of surfactant may allow the surfactant to wash off from the fibers and undesirably interfere with the ability of the composite to transport fluid, or may adversely affect the attachment strength of the absorbent composite 44 to the absorbent article 20. Where the surfactant is compounded or otherwise internally added to the elastomeric polymer, an excessively high level of surfactant can create conditions that cause a poor formation of the polymer fibers.

In some configurations, the surfactant can include at least one material selected from the group that includes polyethylene glycol ester condensates and alkyl glycoside surfactants. For example, the surfactant can be a GLUCOPON surfactant, available from Cognis Corporation, a business having offices located in Cincinnati, Ohio, U.S.A, which can be composed of 40-percent water, and 60-percent d-glucose, decyl, octyl ethers and oligomerics.

In a particular aspect of the invention, the surfactant is in the form of a sprayed-on surfactant comprising a water/surfactant solution which includes 16 liters of hot water (about 45° C. to 50° C.) mixed with 0.20 kg of GLUCOPON 220 UP surfactant available from Cognis Corporation and 0.36 kg of AHCHOVEL Base N-62 surfactant available from Uniqema, a business having offices located in New Castle, Del., U.S.A. When employing a sprayed-on surfactant, a relatively lower amount of sprayed-on surfactant may be desirable to provide the desired containment of the superabsorbent material. Excessive amounts of the fluid surfactant may hinder the desired attachment of the superabsorbent material to the molten, elastomeric meltblown fibers, for example.

An example of an internal surfactant or wetting agent that can be compounded with the elastomeric fiber polymer can include a MAPEG DO 400 PEG (polyethylene glycol) ester, available from BASF, a business having offices located in Freeport, Tex., U.S.A. Other internal surfactants can include a polyether, a fatty acid ester, a soap or the like, as well as combinations thereof.

The components of the absorbent composite 44 can be formed using methods known in the art. While not being limited to the specific method of manufacture, the absorbent composite can utilize a meltblown process and can further be formed on a coform line. Exemplary meltblown processes are described in various patents and publications, including NRL Report 4364, “Manufacture of Super-Fine Organic Fibers” by V. A. Wendt, E. L. Boone and C. D. Fluharty; NRL Report 5265, “An Improved Device For the Formation of Super-Fine Thermoplastic Fibers” by K. D. Lawrence, R. T. Lukas and J. A. Young; and U.S. Pat. No. 3,849,241, to Buntin et al. and U.S. Pat. No. 5,350,624 to Georger et al., all of which are incorporated herein by reference in a manner consistent with the present disclosure. To form “coform” materials, additional components are mixed with the meltblown fibers as the fibers are deposited onto a forming surface. For example, superabsorbent particles and/or staple fibers such as wood pulp fibers may be injected into the meltblown fiber stream so as to be entrapped and/or bonded to the meltblown fibers. Exemplary coform processes are described in U.S. Pat. No. 4,100,324 to Anderson et al.; U.S. Pat. No. 4,587,154 to Hotchkiss et al.; U.S. Pat. No. 4,604,313 to McFarland et al.; U.S. Pat. No. 4,655,757 to McFarland et al.; U.S. Pat. No. 4,724,114 to McFarland et al.; U.S. Pat. No. 4,100,324 to Anderson et al.; and U.K. Patent GB 2,151,272 to Minto et al., all of which are incorporated herein by reference in a manner that is consistent with the present disclosure. Absorbent, elastomeric meltblown webs containing high amounts of superabsorbent are described in U.S. Pat. No. 6,362,389 to D. J. McDowall, and absorbent, elastomeric meltblown webs containing high amounts of superabsorbent and low superabsorbent shake-out values are described in pending U.S. patent application Ser. No. 10/883174 to X. Zhang et al., all of which are incorporated herein in a manner that is consistent with the present disclosure.

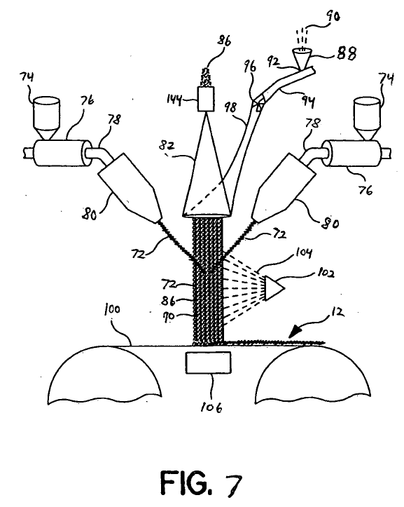

One example of a method of forming the absorbent core 12 of the present invention is illustrated in FIG. 7. The dimensions of the apparatus in FIG. 7 are described herein by way of example. Other types of apparatus having different dimensions and/or different structures may also be used to form the absorbent core 12. As shown in FIG. 7, elastomeric material 72 in the form of pellets can be fed through two pellet hoppers 74 into two single screw extruders 76 that each feed a spin pump 78. The elastomeric material 72 may be a multicomponent elastomer blend available under the trade designation KRATON® G2755 from Kraton Inc., as well as others mentioned above. Each spin pump 78 feeds the elastomeric material 72 to a separate meltblown die 80. Each meltblown die 80 may have 30 holes per inch (hpi). The die angle may be adjusted anywhere between 0 and 70 degrees from horizontal, and is suitably set at about 45 degrees. The forming height may be at a maximum of about 16 inches, but this restriction may differ with different equipment.

A chute 82 having a width of about 24 inches wide may be positioned between the meltblown dies 80. The depth, or thickness, of the chute 82 may be adjustable in a range from about 0.5 to about 1.25 inches, or from about 0.75 to about 1.0 inch. A picker 144 connects to the top of the chute 82. The picker 144 is used to fiberize the pulp fibers 86. The picker 144 may be limited to processing low strength or debonded (treated) pulps, in which case the picker 144 may limit the illustrated method to a very small range of pulp types. In contrast to conventional hammermills that use hammers to impact the pulp fibers repeatedly, the picker 144 uses small teeth to tear the pulp fibers 86 apart. Suitable pulp fibers 86 for use in the method illustrated in FIG. 7 include those mentioned above, such as SULFATATE HJ.

At an end of the chute 82 opposite the picker 144 is a superabsorbent material feeder 88. The feeder 88 pours superabsorbent material 90 into a hole 92 in a pipe 94 which then feeds into a blower fan 96. Past the blower fan 96 is a length of 4-inch diameter pipe 98 sufficient for developing a fully developed turbulent flow at about 5000 feet per minute, which allows the superabsorbent material 90 to become distributed. The pipe 98 widens from a 4-inch diameter to the 24-inch by 0.75-inch chute 82, at which point the superabsorbent material 90 mixes with the pulp fibers 86 and the mixture falls straight down and gets mixed on either side at an approximately 45-degree angle with the elastomeric material 72. The mixture of superabsorbent material 90, pulp fibers 86, and elastomeric material 72 falls onto a wire conveyor 100 moving from about 14 to about 35 feet per minute. However, before hitting the wire conveyor 100, a spray boom 102 optionally sprays an aqueous surfactant mixture 104 in a mist through the mixture, thereby rendering the resulting absorbent core 12 wettable. The surfactant mixture 104 may be a 1:3 mixture of GLUCOPON 220 UP and AHCOVEL Base N-62, available from Cognis Corp. and Uniqema, respectively. An under wire vacuum 106 is positioned beneath the conveyor 100 to assist in forming the absorbent core 12.

While not being limited to the specific method of manufacture, meltblown fibrous nonwoven webs have been found to work particularly well for the stretchable core wrap 14. The general manufacture of such meltblown fibrous nonwoven webs are known in the art. See for example, the previously mentioned meltblown patents referred to above. The fibers may be hydrophilic or hydrophobic, though it is desirable that the resultant web/core wrap be hydrophilic. As referenced above, the fibers may be treated to be hydrophilic such as by the use of a surfactant.

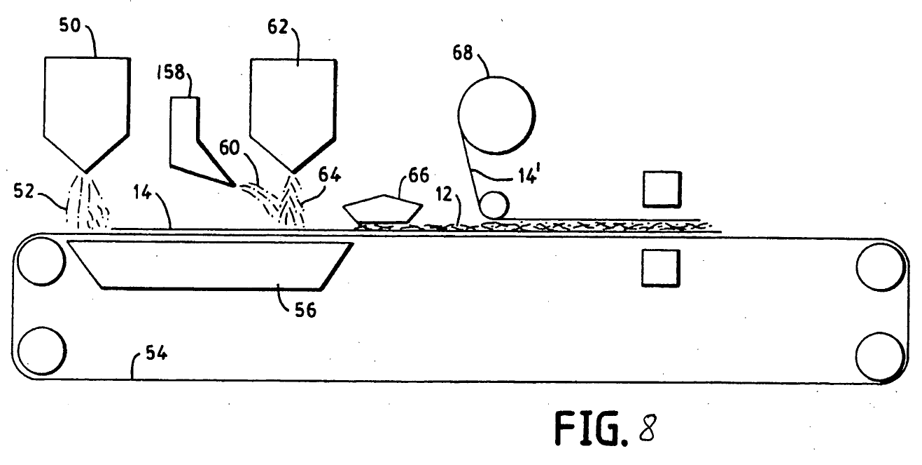

The core wrap 14 of the present invention may also be formed by a process similar to that schematically depicted in FIG. 7. Alternatively, the components of the absorbent composite 44 can be formed inline as a single process. One example of a method of forming the absorbent core 12 and the core wrap 14 of the present invention in a single process is illustrated in FIG. 8. First a web must be formed using a fiber forming apparatus 50 which, in this case, is a meltblown apparatus. In this particular example, as shown in FIG. 8, the meltblown core wrap 14 is formed in-line, however, it is also possible to form the core wrap 14 off-line (such as with the apparatus described in FIG. 7) and then feed it into the process of FIG. 8 in roll form. Returning to FIG. 8, a molten thermoplastic polymer such as a polyolefin is heated and then extruded through a die tip to form a plurality of molten streams of polymer. As the streams of polymer leave the die tip of the meltblown apparatus 50, they are attenuated by high velocity air which draws the molten streams into a plurality of fibers 52 which are deposited onto a forming surface 54 in a random entangled web to form the core wrap 14. To further assist in the web formation and to impart better hold-down of the web onto the forming surface 54, a vacuum 56 may be used underneath the foraminous forming surface 54.

Once the absorbent core wrap 14 has been formed on the forming surface 54 or unrolled from a preformed roll (not shown), the absorbent core 12 can also be formed or deposited in-line onto the surface of the absorbent core wrap 14. As further shown in FIG. 8, there is a source 158 of superabsorbent or other type particles 60 and optionally source 62 of absorbent fibers 64 such as, for example, wood pulp fibers or meltblown fibers or hot melt adhesives for improved containment of superabsorbent materials within the composite. If both superabsorbent materials 60 and the other materials such as absorbent fibers or hot melt adhesives are to be used to form the absorbent core 12, they may be intermixed before they are deposited onto the absorbent core wrap 14 as shown in FIG. 8 or they may be layered so as to sandwich the superabsorbent materials within the interior of the absorbent composite 44. Again to further assist in the deposition and retention of the absorbent core materials onto the surface of the absorbent core wrap 14, the same vacuum source 56 or a separate source if so desired may be used. Optionally, as illustrated in FIGS. 6 and 7, a second core wrap 14′ can be placed on top of the absorbent core 12, such as to sandwich the core between two core wrap layers.

After the absorbent core 12 has been deposited onto the absorbent core wrap 14, the core wrap 14 can be at least partially sealed around the absorbent core 12 so as to partially envelope the absorbent core 12 to form the absorbent composite 44. As shown in FIGS. 3-6, to completely envelope the entire absorbent core 12, the core wrap 14 can completely wrap around the core 12 and be sealed, either to itself or to the core itself using conventional means known in the art, including but not limited to, adhesive, heat, pressure, ultrasonic, aperturing, and autogenous. It may also be desirable that the ends of the absorbent composite 44 be sealed. Due to the thermoplastic nature of the fibers of the core wrap 14, the core wrap 14 may be heat sealed to itself thus avoiding the need for glue though glue and/or other methods of bonding mentioned above can also be used if so desired. In addition, if so desired, the absorbent core materials 60 and 64 may be cycled on and off so that end seals can be formed in between the deposits of core material. Further, if the absorbent fibers 64 are also thermoplastic in nature, end and side seals can be made in the core wrap 14 which bond right through the absorbent core 12.

The present invention may be better understood with reference to the following examples.

### EXAMPLES

### Example 1

A stretchable meltblown core wrap having a basis weight of 8 gsm was prepared according to the present invention using a coform process such as depicted in FIG. 7, but without the pulp stream. The following machine settings were utilized:
the line speed was 122 feet per minute
the die tip-to-wire forming height was 10.5 inches
the, die angle was 45 degrees
the die to die distance was 4 inches
the polymer output rate was 151 g/min
the die primary air temperature was 740 degrees F (393° C.)
   The polymer utilized was a 60 melt flow rate (MFR) VISTAMAXX 2210 treated with 400 ppm of Peroxide.

During the process, the fibrous web was treated with a 3:1 mass ratio AHCHOVEL Base N-62/GLUCOPON 220 UP surfactant at an add-on rate of 0.16% by weight. The resulting core wrap was then tested for various properties, the results of which can be seen in Tables 1-2 below. It can be seen that the resulting core wrap had mean flow pore diameter of 34.7 microns with a standard deviation of 7.2 as measured by the Mean Flow Pore Diameter Test as described below. The core wrap had an average MD elongation of 68.9% (576.5 gram-biasing force) and an average CD elongation of 390.9% (163.8 gram-biasing force) using the Elongation Test as described below. The average fiber diameter was 5.9 μm using the Fiber Diameter test as described below. The MD Peak Energy was 1.6 inch-pound (1787 cm-g) and CD Peak Energy was 3.0 inch-pound (3402 cm-g). The Air Permeability was about 3495 m³/m²/min using the Air Permeability Test as described below. Furthermore, the sample had an Elastic Recovery of about 94.5% in the MD and 23.2% in the CD using the Elastic Recovery Test below. Additional information regarding Elastic Recovery can be seen in Table 3 below.

### Example 2

A stretchable meltblown core wrap with a basis weight of 10 gsm was prepared using the same process and polymer as in Example 1 above, except that the line speed was reduced to about 98 feet per minute. The AHCOVEL/GLUCON surfactant add-on was increased to about 0.19% by weight. The resulting core wrap was then tested for various properties, the results of which can be seen in Tables 1-2 below. It can be seen that the resulting core wrap had mean flow pore diameter of 26.9 microns with a standard deviation of 2.0. The core wrap had an average MD and CD elongations of 61.4% and 410.8%, respectively when biasing forces of 765.5 grams and 207.3 grams were applied to the sample in the respective directions. The MD and CD Peak Energies were 2.0 and 4.0 inch-pounds, respectively. Furthermore, the core wrap had an Elastic Recovery of about 93.6% in the MD and 25.8% in the CD. Additional information regarding Elastic Recovery can be seen in Table 3 below.

### Example 3

A stretchable meltblown core wrap with a basis weight of about 15 gsm was prepared using the same process and polymer as in Example 1 except that the line speed was reduced to about 65 feet per minute. The AHCOVEL/GLUCON surfactant add-on increased to about 0.35% by weight. The resulting core wrap was then tested for various properties, the results of which can be seen in Tables 1-2 below. It can be seen that the resulting core wrap had mean flow pore diameter of 22.1 microns with a standard deviation of 4.8 microns. The core wrap had average MD and CD elongations of 63.5% and 346.1%, respectively when biasing forces of 1203.6 grams and 280.2 grams were applied to the sample in the respective directions. The MD and CD Peak Energies were 3.3 and 4.5 inch-pounds, respectively. The Air Permeability was about 1499 m³/m²/min. Furthermore, the core wrap had an Elastic Recovery of about 94.5% in the MD and 60.2% in the CD. Additional information regarding Elastic Recovery can be seen in Table 3 below.

### Example 4

A stretchable meltblown core wrap with a basis weight of about 20 gsm was prepared using the same process and polymer as in Example 1 except that the line speed was reduced to about 49 feet per minute. The AHCOVEL/GLUCON surfactant add-on was about 0.30% by weight. The resulting core wrap was then tested for various properties, the results of which can be seen in Tables 1-2 below. It can be seen that the resulting core wrap had mean flow pore diameter of 15.6 microns with a standard deviation of 0.7. The core wrap had average MD and CD elongations of 64.1% and 379.6%, respectively when biasing forces of 1608.2 grams and 431.0 grams were applied to the sample in the respective directions. The core wrap had an average fiber diameter of about 5.69 μm. The Air Permeability was about 905 m³/m²/min. The MD and CD Peak Energies were 4.6 and 7.6 inch-pounds, respectively. Furthermore, the core wrap had an Elastic Recovery of about 95.2% in the MD and 52.4% in the CD. Additional information regarding Elastic Recovery can be seen in Table 3 below.

### Example5

A stretchable meltblown core wrap with a basis weight of about 30 gsm was prepared using the same process and polymer as in Example 1 except that the line speed was reduced to about 32 feet per minute. The AHCOVEL/GLUCON surfactant add-on was about 0.35% by weight. The resulting core wrap was then tested for various properties, the results of which can be seen in Tables 1-2 below. It can be seen that the resulting core wrap had mean flow pore diameter of 14.2 microns with a standard deviation of 1.0. The core wrap had average MD and CD elongations of 65.0% and 356.4%, respectively when biasing forces of 2574 grams and 576 grams were applied to the sample in the respective directions. The MD and CD Peak Energies were 7.3 and 9.6 inch-pounds, respectively. Furthermore, the core wrap had an Elastic Recovery of about 95.5% in the MD and 65.7% in the CD. Additional information regarding Elastic Recovery can be seen in Table 3 below.

### Example6

A stretchable meltblown core wrap with a basis weight of about 50 gsm was prepared using the same process and polymer as in Example 1 except that the line speed was reduced to about 26 feet per minute. The AHCOVEL/GLUCON surfactant add-on was about 0.65% by weight. The resulting core wrap was then tested for various properties, the results of which can be seen in Tables 1-2 below. It can be seen that the resulting core wrap had mean flow pore diameter of 9.3 microns with a standard deviation of 0.4. The core wrap had average MD and CD elongations of 103.8% and 488.0%, respectively when biasing forces of 33082 grams and 1151 grams were applied to the sample in the respective directions. The MD and CD Peak Energies were 25.5 and 37.6 inch-pounds, respectively. The Air Permeability was about 235m³/m²/min. Furthermore, the core wrap had an Elastic Recovery of about 92.3% in the MD and 51.4% in the CD. Additional information regarding Elastic Recovery can be seen in Table 3 below.

### Example7

A stretchable meltblown core wrap with a basis weight of about 80 gsm was prepared using the same process and polymer as in Example 1 except that the line speed was reduced to about 24 feet per minute. The AHCOVEL/GLUCON surfactant add-on was about 0.44% by weight. The resulting core wrap was then tested for various properties, the results of which can be seen in Tables 1-2 below. It can be seen that the resulting core wrap had mean flow pore diameter of 7.8 microns with a standard deviation of 0.7. The core wrap had average MD and CD elongations of 93.5% and 450.3%, respectively when biasing forces of 5787 grams and 1689 grams were applied to the sample in the respective directions. The MD and CD Peak Energies were 34.8 and 69.6 inch-pounds, respectively. The core wrap had an average fiber diameter of about 5.38 μm. Furthermore, the core wrap had an Elastic Recovery of about 90.0% in the MD and 57.7% in the CD. Additional information regarding Elastic Recovery can be seen in Table 3 below.

### Example8

A stretchable meltblown core wrap with a basis weight of about 100 gsm was prepared using the same process and polymer as in Example 1 except that the line speed was reduced to about 20 feet per minute. The AHCOVEL/GLUCON surfactant add-on was about 0.94% by weight. The resulting core wrap was then tested for various properties, the results of which can be seen in Tables 1-2 below. It can be seen that the resulting core wrap had mean flow pore diameter of 9.7 microns with a standard deviation of 0.1. The core wrap had average MD and CD elongations of 95.0% and 620.9%, respectively when biasing forces of 7739 grams and 2219 grams were applied to the sample in the respective directions. The MD and CD Peak Energies were 4.5 and 5.0 inch-pounds, respectively. Furthermore, the core wrap had an Elastic Recovery of about 88.6% in the MD and 33.9% in the CD. Additional information regarding Elastic Recovery can be seen in Table 3 below.

TABLE 1
Elongation and Cycle Elastic Recovery Test Data
Core Wrap: Mean Flow Pore Diameter, % Elongation and Elastic Recovery

| | | Mean | | | | | | | |
|---|---|---|---|---|---|---|---|---|---|
| | | Flow Pore | | | | | | M.D. | C.D. |
| | | Diameter | | Max M.D. | Max C.D. | Max M.D. | Max C.D. | Elastic | Elastic |
| Sample | B.W. | (microns) | | Elongation | Elongation | Load | Load | Recovery | Recovery |
| I.D. | gsm | AVG | STD | Percent(%) | Percent(%) | (gf) | (gf) | (%) | (%) |
| Example 1 | 8 | 34.7 | 7.2 | 68.9 | 390.9 | 576.5 | 163.8 | 94.5 | 23.2 |
| Example 2 | 10 | 26.9 | 2.0 | 61.4 | 410.8 | 765.5 | 207.3 | 93.6 | 25.8 |
| Example 3 | 15 | 22.1 | 4.8 | 63.5 | 346.1 | 1203.6 | 280.2 | 94.5 | 60.2 |
| Example 4 | 20 | 15.6 | 0.7 | 64.1 | 379.6 | 1608.2 | 431.0 | 95.2 | 52.4 |
| Example 5 | 30 | 14.2 | 1.0 | 65.0 | 356.3 | 2573.5 | 575.6 | 95.5 | 65.7 |
| Example 6 | 50 | 9.3 | 0.4 | 103.8 | 488.0 | 3081.9 | 1151.3 | 92.3 | 51.4 |
| Example 7 | 80 | 7.8 | 0.7 | 93.5 | 450.3 | 5786.6 | 1689.2 | 90.0 | 57.7 |
| Example 8 | 100 | 9.7 | 0.1 | 95.0 | 620.9 | 7738.5 | 2218.9 | 88.6 | 33.9 |

TABLE 2
Core Wrap Fiber Diameter, Air Permeability and Surfactant Add-on

| | | Average | | | |
|---|---|---|---|---|---|
| | | Fiber | | Ahcovel/ | Ahcovel/ |
| | Basis | Diameter | | Glucopon | Air |
| Sample | Weight | μm | | Add-on | Permeability |
| I.D. | gsm | AVG | STD | (%) | M\hat{ }3/M\hat{ }2/Min |
| Example 1 | 8 | 5.86 | 0.72 | 0.1631 | 3495 |
| Example 2 | 10 | | | 0.1911 | |
| Example 3 | 15 | | | 0.3493 | 1499 |
| Example 4 | 20 | 5.69 | 0.52 | 0.2966 | 905 |
| Example 5 | 30 | | | 0.3488 | |
| Example 6 | 50 | | | 0.6456 | 235 |
| Example 7 | 80 | 5.38 | 1.11 | 0.4393 | |
| Example 8 | 100 | | | 0.9415 | |

TABLE 3
Cycle Elastic Recovery Test Data
Extension/Retraction Loads (gram-force)

| | Basis Weights (gsm) | | | | | | | |
|---|---|---|---|---|---|---|---|---|
| | 8 | 10 | 15 | 20 | 30 | 50 | 80 | 100 |
| % Elongation | MD Load to Elongate (gf) | | | | | | | |
| 10% | 214.4 | 239.7 | 415.5 | 576.3 | 935.1 | 1026.6 | 1612.4 | 2124.8 |
| 20% | 392.1 | 467.1 | 776.5 | 1081.0 | 1752.5 | 1862.5 | 3026.6 | 3946.3 |
| 30% | 499.5 | 618.4 | 1003.8 | 1399.8 | 2282.9 | 2383.1 | 4012.9 | 5339.4 |
| 40% | | | | | | 2702.9 | 4618.8 | 6268.3 |
| Energy Loading(g * cm) | 470 | 556 | 1038 | 1444 | 2355 | 5008 | 7107 | 9507 |
| % Retraction | MD Load to Retract(gf) | | | | | | | |
| 10% | 33.7 | 32.9 | 49.5 | 71.0 | 123.6 | 53.3 | 16.0 | 0.2 |
| 20% | 155.1 | 178.7 | 247.2 | 347.0 | 580.8 | 249.6 | 406.9 | 485.8 |
| 30% | 414.6 | 512.3 | 711.8 | 996.8 | 1626.8 | 501.5 | 1156.7 | 1529.0 |
| Energy UnLoading(g * cm) | 206 | 245 | 429 | 601 | 999 | 1693 | 2507 | 3350 |
| % set | 5.5 | 6.4 | 5.5 | 4.8 | 4.5 | 7.7 | 10.0 | 11.4 |
| % Hyterisis Loss | 56.1 | 56.1 | 58.7 | 58.4 | 57.6 | 66.2 | 64.7 | 64.8 |
| % Elongation | CD Load to Elongate(gf) | | | | | | | |
| 10% | 20.8 | 25.5 | 40.6 | 52.7 | 97.6 | 162.9 | 283.6 | 336.1 |
| 20% | 35.8 | 45.8 | 71.8 | 96.4 | 169.3 | 287.6 | 492.9 | 593.1 |
| 30% | 48.2 | 62.5 | 95.4 | 128.3 | 221.0 | 379.7 | 643.0 | 784.0 |
| 40% | 57.7 | 75.4 | 114.6 | 153.3 | 259.9 | 448.1 | 753.7 | 926.1 |
| 50% | 65.9 | 86.1 | 130.4 | 173.7 | 290.8 | 503.2 | 841.6 | 1038.9 |
| 60% | 72.9 | 95.2 | 144.4 | 192.0 | 317.9 | 550.7 | 917.0 | 1136.1 |
| 70% | 79.5 | 103.8 | 156.5 | 208.1 | 342.6 | 593.9 | 984.4 | 1223.5 |
| 80% | 85.8 | 111.5 | 167.5 | 222.7 | 365.8 | 633.6 | 1045.6 | 1303.6 |
| Energy Loading(g * cm) | 865 | 1225 | 1382 | 2162 | 3176 | 8640 | 12942 | 25429 |
| % Retraction | CD Load to Retract(gf) | | | | | | | |
| 10% | 4.4 | 4.3 | 4.6 | 5.0 | 4.9 | 5.5 | 5.5 | 5.7 |
| 20% | 4.0 | 4.4 | 4.9 | 4.8 | 5.5 | 5.3 | 5.4 | 5.6 |
| 30% | 3.9 | 4.3 | 5.4 | 4.9 | 6.1 | 5.2 | 5.6 | 5.7 |
| 40% | 5.0 | 5.3 | 10.2 | 7.1 | 15.4 | 5.2 | 6.2 | 5.6 |
| 50% | 6.5 | 7.0 | 14.5 | 11.5 | 24.7 | 11.8 | 26.3 | 5.8 |
| 60% | 8.2 | 9.3 | 18.6 | 15.7 | 32.8 | 22.3 | 47.7 | 5.4 |
| 70% | 9.6 | 11.5 | 22.6 | 20.1 | 41.0 | 32.8 | 68.2 | 16.1 |
| 80% | 11.4 | 13.1 | 27.4 | 24.4 | 50.3 | 43.8 | 87.9 | 34.0 |
| Energy UnLoading(g * cm) | 224 | 312 | 396 | 522 | 841 | 1884 | 3014 | 5024 |
| % set | 76.8 | 69.1 | 39.8 | 47.6 | 33.9 | 48.6 | 42.3 | 66.1 |
| % Hyterisis Loss | 74.1 | 74.6 | 71.4 | 75.9 | 73.9 | 78.2 | 76.7 | 80.3 |

Test Procedures
Fiber Diameter Test

The fibers of sample nonwoven webs were sputter coated with gold using DENTON DESK II sputter coater (available from Denton Vacuum, a business having offices located in Moorestown, N.J. U.S.A.) to a gold thickness of about 400 to 500 Angstroms. The fibers were then examined using a Scanning Electron Microscope (SEM) such as a JOEL JSM-840, available from Jeol USA, Inc., a business having offices located in Peabody, Mass. U.S.A. One hundred fibers were selected at random and individual fiber diameters were measured using the electronic cursors of the SEM. Particular care should be taken not to select fibers which have been fused together.

Mean Flow Pore Diameter Test

The average pore size and maximum pore size were measured using a CFP 1100AEXLH Automated Capillary Flow Porometer available from PMI Inc., a business having offices located in Ithaca, N.Y. U.S.A. Using a maximum pressure of 75 psi and a maximum flow 150,000 cc/m, a 38 mm specimen was placed in the specimen holder. The specimen was placed in the reservoir and the top was tightened to retain the specimen in the retaining area. The test was started with a dry run. When the dry run was completed, the specimen was immersed in SILWICK silicone oil wetting agent having a surface tension of 20.1 dynes/cm (available from Dow Chemical Company, a business having offices located in Freeport, Tex. U.S.A.). The specimen was then placed back into the holder, the top was tightened and the wet run was started. The results were reported as the smallest detected pore pressure, the smallest detected pore diameter, the mean flow pore pressure, the mean flow pore diameter, the bubble point pressure, the bubble point pore diameter, the maximum pore size distribution and the diameter at maximum pore size distribution.

Air Permeability Test

This test measures the rate and volume of air flow through a sample under a prescribed surface pressure differential. Under controlled conditions, a suction fan drew air through a known area of the sample. The air flow rate was adjusted to a prescribed pressure differential. The results were expressed as the rate of air flow in cubic feet per minute (ft³/min), which when divided by the sample test area gives the air flow rate per unit area of the sample.

Air flow rate and volume are an indication of fabric breathability. The air permeability test procedure used for the present invention is comparable to INDA 70.1 and ASTM D737-96 Industry Tests. The test was performed using a TEXTEST FX 3300 available from Textest Ltd, Zurich, Switzerland. A 6×6 inch sample was clamped under the test head with a sample test area of 38 cm². The range was adjusted until the pressure stabilized to 125 Pa, indicated by a green light on the display. The air flow rate value was then reported in CFM (ft³/min). To convert from CFM to m³/m²/min, multiply by 7.4527. Results are reported as an average of five specimens.

Elongation Test

This test measures the peak (maximum) load and the corresponding percent elongation (strain) at the peak load of a sample. It measures the load (strength) in grams and elongation in percent. A SINTECH 2 tensile tester (available from Sintech Corporation, a business having offices located in Cary, N.C. U.S.A.), an INSTRON TM tensile tester (available from the Instron Corporation, a business having offices located in Canton, Mass. U.S.A.), a THWING-ALBERT INTELLECT II tensile tester (available from the Thwing-Albert Instrument Co., a business having offices located in Philadelphia, Pa. U.S.A) or a SYNERGIE 200 tensile tester (available from MTS Systems Corporation, a business having offices located in Eden Prairie, Minn. U.S.A.) may be used for this test. The samples for the present invention were performed using the SYNERGIE 200 tensile tester.

To perform the test, samples were cut to a size of 3 inches by 6 inches, (76 mm×152 mm). The samples were placed into the two clamps on the SYNERGIE 200, each having two jaws with a face size of 1 inch high by 3 inches wide (25 mm×76 mm) each, such that each jaw was in facing contact with the sample and which held the material in the same plane, separated by 51 mm. The jaws then moved apart at a constant rate of extension of 300 mm/min until the samples broke. The results were obtained as an average of five specimens in both the machine direction (MD) and the cross-machine direction (CD).

The results that can be obtained are the maximum (peak) strain or elongation in percent and the maximum (peak) load in gram-force needed to reach the maximum elongation. The test is therefore a destructive test which allows determination of the maximum extensibility or stretch of a sample specimen and the force or load required to achieve that maximum extensibility. The peak energy is the calculated area under the elongation-load curve from the origin to the point of rupture.

Cycle Elastic Recovery Test

The same SYNERGIE 200 instrument as described above in the Elongation Test was again used to perform the Cycle Elastic Recovery Test. However, the gauge length was set at 51 mm and the jaw speed was changed to 508 mm/min. The samples were cut from the same materials used in the cut strip tensile test. Five specimens were tested for each material sample.

In the Cycle Elastic Recovery Test, the samples were not pulled to the maximum elongation point of rupture. Instead, the samples were extended to a peak strain equal to 50% of the average peak strain determined in the Elongation Test. The loads (gram-force) required to extend and retract the samples 10%, 20%, 30%, 40% and in some instances 50%, 60% and 80% were determined on extension and retraction curves. Each test was performed as a 1-cycle test.

To perform the test, samples were cut to a size of 3 inches by 6 inches, (76 mm×152 mm). The samples were placed into the two clamps on the SYNERGIE 200, each having two jaws with a face size of 1 inch high by 3 inches wide (25 mm×76 mm) each, such that each jaw was in facing contact with the sample and which held the material in the same plane, separated by 51 mm. The jaws then moved apart at a constant rate of extension of 508 mm/min until the specified load was reached. The samples were then allowed to retract. The results were obtained as an average of five specimens in both the machine direction (MD) and the cross-machine direction (CD).

Often, stretchable materials do not recover or retract to their original length when the extending load is removed. The amount of length not recovered is referred to as “percent set (% set)” and is defined as the set or strain at which the force value reaches 10 grams on the retraction curve. The % set is calculated as a percent strain from the 10-gram load point on the retraction curve to the return point on the retraction curve. To calculate “percent recovery,” the formula (100-% set) is used. For example, if percent set is 5.5%, the percent recovery is (100-5.5)=94.5%, meaning that the sample was able to recover 94.5% of the extended length. The Cycle Elastic Recovery Test procedure also gives an elastic material property known as % Hysteresis Loss calculated as [(Energy loading)-(Energy unloading)/Energy Loading]×100.

It will be appreciated that details of the foregoing examples, given for purposes of illustration, are not to be construed as limiting the scope of this invention. Although only a few exemplary embodiments of this invention have been described in detail above, those skilled in the art will readily appreciate that many modifications are possible in the examples without materially departing from the novel teachings and advantages of this invention. For example, features described in relation to one example may be incorporated into any other example of the invention.

Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention. As various changes could be made in the above constructions without departing from the scope of the invention, it is intended that all matter contained in the above description shall be interpreted as illustrative and not in a limiting sense.

What is claimed is:

1. An absorbent article comprising:
a stretchable backsheet; and
an absorbent composite in facing relationship with said stretchable backsheet comprising an absorbent core and a stretchable core wrap that at least partially envelopes said absorbent core;
wherein said absorbent core includes a quantity of superabsorbent materials; and
wherein said core wrap has a mean flow pore diameter less than about 41 microns.

2. The absorbent article of claim 1 wherein said backsheet is elastically extensible.

3. The absorbent article of claim 1 further comprising a stretchable bodyside liner in facing relationship with said absorbent composite to sandwich said absorbent composite between said bodyside liner and said backsheet.

4. The absorbent article of claim 3 wherein said stretchable bodyside liner is elastically extensible.

5. The absorbent article of claim 1 wherein said absorbent core comprises a matrix comprising at least polymer fibers.

6. The absorbent article of claim 1 wherein said absorbent core is stretchable.

7. The absorbent article of claim 1 wherein said absorbent core is elastically extensible.

8. The absorbent article of claim 1 wherein said stretchable core wrap is elastically extensible.

9. The absorbent article of claim 1 wherein said absorbent core comprises at least about 60% by weight superabsorbent materials.

10. The absorbent article of claim 1 wherein said absorbent core comprises at least about 80% by weight superabsorbent materials.

11. The absorbent article of claim 1 wherein said absorbent core further comprises absorbent fiber.

12. The absorbent article of claim 1 wherein said stretchable core wrap is attached to said stretchable backsheet by an attachment means selected from the group consisting of ultrasonic, pressure, adhesive, aperturing, heat, sewing thread or strand, autogenous, hook-and-loop, and combinations thereof.

13. The absorbent article of claim 1 wherein said mean flow pore diameter of said stretchable core wrap is less than about 35 microns.

14. The absorbent article of claim 1 wherein said mean flow pore diameter of said stretchable core wrap is in the range of about 8 to about 35 microns.

15. The absorbent article of claim 1 wherein said stretchable core wrap has an air permeability greater than about 200 m³/m²/min.

16. The absorbent article of claim 1 wherein said stretchable core wrap has an air permeability between about 200 and about 3500 m³/m²/min.

17. The absorbent article of claim 1 wherein said stretchable core wrap comprises elastomeric polymer fibers.

18. The absorbent article of claim 1 wherein said stretchable core wrap comprises fibers having a fiber diameter less than about 20 microns.

19. The absorbent article of claim 1 wherein said stretchable core wrap comprises fibers having a fiber diameter less than about 8 microns.

20. The absorbent article of claim 19 wherein said fibers comprise 80% of said stretchable core wrap.

21. The absorbent article of claim 1 wherein said stretchable core wrap comprising fibers having a fiber diameter less than about 7 microns.

22. The absorbent article of claim 21 wherein said fibers comprise at least about 95% of said stretchable core wrap.

23. The absorbent article of claim 1 wherein said stretchable core wrap comprises a nonwoven web selected from the group consisting of meltblown, spunbond, spunlace, spunbond-meltblown-spunbond, coform, and combinations thereof.

24. The absorbent article of claim 1 wherein said stretchable core wrap comprises absorbent materials.

25. The absorbent article of claim 1 wherein said stretchable core wrap has an elongation in at least the machine direction of less than about 104% when a biasing force of about 3100 gram-force is applied in said machine direction.

26. The absorbent article of claim 1 wherein said stretchable core wrap has an elongation in at least a cross-machine direction of less than about 621% when a biasing force of about 2300 gram-force is applied in said cross-machine direction.

27. The absorbent article of claim 1 wherein said stretchable core wrap has an elastic recovery of between about 89% and about 95% in a machine direction.

28. The absorbent article of claim 1 wherein said stretchable core wrap has an elastic recovery of between about 23% and about 66% in a cross-machine direction.

29. The absorbent article of claim 1 wherein said stretchable core wrap is hydrophilic.

30. The absorbent article of claim 29 wherein said stretchable core wrap is treated with a surfactant.

31. The absorbent article of claim 1 wherein said stretchable core wrap comprises a hydrophilicity boosting composition having a quantity of nanoparticles, wherein said nanoparticles have a particle size of from about 1 to about 750 nanometers.

32. The absorbent article of claim 31 wherein said nanoparticles are selected from the group consisting of titanium dioxide, layered clay minerals, alumina oxide, silicates, and combinations thereof.

33. An absorbent article comprising:
a stretchable backsheet;
a stretchable bodyside liner
an absorbent composite disposed between said stretchable backsheet and said stretchable bodyside liner comprising an elastically extensible absorbent core and an elastically extensible core wrap having a mean flow pore diameter less than about 35 microns;
wherein said elastically extensible absorbent core includes at least 60% superabsorbent materials;
wherein said elastically extensible core wrap comprises fibers having a fiber diameter of 20 microns or less; and
wherein said elastically extensible core wrap at least partially envelopes said elastically extensible absorbent core.

34. An absorbent article comprising:
A stretchable backsheet;
A stretchable bodyside liner;
An absorbent composite disposed between said stretchable backsheet and said stretchable bodyside liner comprising a stretchable absorbent core and a stretchable absorbent core wrap;
wherein said stretchable absorbent core comprises at least about 60% of a superabsorbent material having a surface cross-linking and a substantially non-covalently bonded surface coating with a partially hydrolysable cationic polymer; and
wherein said stretchable core wrap has a mean flow pore diameter less than about 35 microns.
